(19) United States
(12) Patent Application Publication
(10) Pub. No.: US 2010/0221758 A1
(43) Pub. Date: Sep. 2, 2010

(54) Sensors for Measuring Contaminants

(75) Inventor: Mitchell C. SANDERS, West Boylston, MA (US)

Correspondence Address: MIRICK, O'CONNELL, DEMALLIE & LOUGEE, LLP, 1700 WEST PARK DRIVE, WESTBOROUGH, MA, 01581, US

(73) Assignee: ECI Biotech Inc., Worcester, MA (US)

(21) Appl. No.: 12/769,511

(22) Filed: Apr. 28, 2010

Related U.S. Application Data
(63) Continuation of application No. PCT/US2008/081483, filed on Oct. 28, 2008.
(60) Provisional application No. 60/983,190, filed on Oct. 28, 2007.

(51) Int. Cl. G01N 33/554; C12M 1/34

(52) U.S. Cl. 435/7.32; 435/287.1; 435/287.9

(57) ABSTRACT

The present invention provides biosensors and methods of use for detecting the presence or absence of mycoplasma contamination through the detection of hydrolytic enzymes that are conserved among Mycoplasma species. Such hydrolytic enzymes include, but are not limited to, proteases, reductases and nucleases.

### RELATED APPLICATION

This application is a continuation of co-pending PCT international patent application PCT/US2008/081483, filed Oct. 28, 2008, the entire contents of which are incorporated by reference for all purposes.

### BACKGROUND OF THE INVENTION

Mycoplasmas are very small microorganisms (Class Mollicutes) without cell walls that can cause infections in humans, animals, and plants. Mycoplasmas are also commonly found contaminating buffer solutions, and tissue culture media used in life science research. The Mycoplasma and Acholeplasma species, Acholeplasma laidlawii, M. hyorhinis, M. orale, M. salivarium, M. arginini, and M. hominis, account for about 98% of the tissue culture contaminants (McGarrity, G. J., & Carson, D. A., Adenosine phosphorylase-mediated nucleoside toxicity. Application towards the detection of mycoplasmal infection in mammalian cell cultures. Exp Cell Res. 1982 May; 139(1):199-205). As used herein, “mycoplasma” or “mycoplasmas” refers generally to members of the Class Mollicutes, including Mycoplasma and Acholeplasma species.

There is a clear unmet need for the real time detection of mycoplasmas for infection control monitoring in hospitals and for quality control of buffers and tissue culture media used in clinical laboratory testing and life science research.

### SUMMARY OF THE INVENTION

The present invention provides biosensors and methods of use for detecting the presence or absence of mycoplasma contamination through the detection of hydrolytic enzymes that are conserved among Mycoplasma species. Such hydrolytic enzymes include, but are not limited to, proteases, reductases and nucleases

In preferred embodiments, the present invention provides a biosensor for detecting the presence or absence of Mycoplasma contamination comprising a support and a detectably labeled substrate for an enzyme produced and/or secreted by a mycoplasma, wherein the substrate is attached to the support. Typically, the enzyme is a Mycoplasma-specific hydrolytic enzyme selected from the group consisting of proteases, reductases and nucleases. In certain preferred embodiments, the enzyme is a Mycoplasma-specific protease selected from the group consisting of the gene product of pepA1 (MCAP_{—}0157), pepA2 (MCAP_{—}0195), pepA (leucyl aminopeptidase, such as MHP7448_{—}0464), MCAP_{—}0267 (metalloendopeptidase), pepP (Xaa-Pro endopeptidase, such as MCAP_{—}0341 or MHP7448_{—}0649), MCAP_{—}0509, mapP (methionine amino peptidase, MCAP_{—}0675 or MHP7448_{—}0173), mixtures thereof and homologous enzymes with at least 40% sequence identity. When the enzyme is a mycoplasma-specific protease, preferred substrates include leucine-(7-methoxycoumarin-4-yl)acetyl (leu-MCA), arginine-(7-methoxycoumarin-4-yl)acetyl (arg-MCA), methionine-(7-methoxycoumarin-4-yl)acetyl (met-MCA), an acetoxymethyl ester or maleimide derivative of blue dye number 1 coupled to a peptide substrate of the mycoplasma-specific protease.

In other preferred embodiments, the enzyme is a Mycoplasma-specific reductase selected from the group consisting of the gene product of nrdE (such as MCAP_{—}0101), MCAP_{—}0427, trxB (thioredoxin reductase, such as MCAP_{—}0779 or MHP7448_{—}0098), MCAP_{—}0858 and mixtures thereof. When enzyme is a mycoplasma-specific reductase, suitable substrates include reactive black 5,5,5′-dithio-bis-(2-nitrobenzoic acid) (DTNB), BODIPY®FL L-cystine, 2′,7′-difluoro-4′-(2-(5-((dimethyl amino phenyl)azo)pyridyl)dithiopropionyl aminomethyl)fluorescein (DFDMAP-fluorescein), or an azo dye that is sensitive to decolorization by microbial reductases.

In yet other preferred embodiments, the enzyme is a mycoplasma-specific nuclease selected from the group consisting of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof. When the enzyme is a mycoplasma-specific nuclease, a preferred substrate is an acetoxymethyl ester or maleimide derivative of blue dye number 1 coupled to an aminoallyl-dNTP labeled nucleic acid substrate of the mycoplasma-specific nuclease. Typically the substrate is a reagent container, a culture medium container or a cell culture container.

In other aspects, the present invention provides a method of detecting mycoplasma contamination of a cell culture comprising the steps of providing a cell-permeable detectable label coupled to a cell-impermeant carrier in the culture medium wherein cleavage of the detectable label by a mycoplasma-specific enzyme is followed by uptake of the detectable label into cells; and detecting labeled cells, thereby detecting mycoplasma contamination of the cell culture. In certain embodiments, the mycoplasma-specific enzyme is a protease and the detectable label is an acetoxymethyl ester of derivative of blue dye number 1 coupled to a peptide substrate of the mycoplasma-specific protease. Preferred proteases can be selected from the group consisting of the gene product of pepA1 (MCAP_{—}0157), pepA2 (MCAP_{—}0195), pepA (leucyl aminopeptidase, such as MHP7448_{—}0464), MCAP_{—}0267 (metalloendopeptidase), pepP (Xaa-Pro endopeptidase, such as MCAP_{—}0341 or MHP7448_{—}0649), MCAP_{—}0509, mapP (methionine amino peptidase, MCAP_{—}0675 or MHP7448_{—}0173), and mixtures thereof. In other preferred embodiments, the mycoplasma-specific enzyme is a nuclease and the detectable label is an acetoxymethyl ester of derivative of blue dye number 1 coupled to a nucleic acid substrate of the mycoplasma-specific nuclease Preferred nucleases can be selected from the group consisting of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof.

In other aspects, the present invention provides a method of determining the presence or absence of mycoplasma in a sample, comprising the steps of contacting the sample with a detectably labeled substrate for an enzyme produced and/or secreted by a mycoplasma under conditions that result in the modification of the substrate by the enzyme; and detecting the modification or the absence of the modification of the substrate wherein modification of the substrate indicates the presence of mycoplasma in the sample, and wherein the absence of modification of the substrate indicates the absence of mycoplasma in the sample. Preferably, the level of the detectable label is quantitatively related to the presence or amount of mycoplasma in the sample.

In preferred embodiments, the enzyme is a hydrolytic enzyme selected from a protease, a nuclease or a reductase. In certain embodiments, enzyme is a protease selected from group consisting of the gene product of pepA1 (MCAP_{—}0157), pepA2 (MCAP_{—}0195), pepA (leucyl aminopeptidase, such as MHP7448_{—}0464), MCAP_{—}0267 (metalloendopeptidase), pepP (Xaa-Pro endopeptidase, such as MCAP_{—}0341 or MHP7448_{—}0649), MCAP_{—}0509, mapP (methionine amino peptidase, such as MCAP_{—}0675 or MHP7448_{—}0173), and mixtures thereof. In other preferred embodiments, the enzyme is a reductase selected from the group consisting of the gene product of nrdE (such as MCAP_{—}0101), MCAP_{—}0427, trxB (thioredoxin reductase, such as MCAP_{—}0779 or MHP7448_{—}0098), MCAP_{—}0858 and mixtures thereof. In yet other preferred embodiments, the enzyme is a nuclease selected from the group consisting of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a photograph of the decolorization of an azo dye, reactive black 5, with supernatants of cultured bacteria. Each well was incubated with 10 μg of reactive black 5 plus 190 μl of culture supernatant from the following bacterium: E. coli, E. faecalis, S. aureus, P. aeruginosa, S. pyogenes, and S. marcescens. Such azo dyes were decolorized by most bacteria after incubation with the dye for about 18 hours. The decolorization is indicative of reductases produced by the bacteria.

FIG. 2A is a diagrammatic illustration of an embodiment of a contamination biosensor 200 placed on a container 100 for a reagent or culture medium.

FIG. 2B a diagrammatic illustration of an embodiment of a contamination biosensor 210 placed on a container 110 for tissue culture.

FIG. 3 is a diagrammatic illustration of an embodiment of a mycoplasma contamination detection system for cell culture, showing in FIG. 3A a cell 300 in an uncontaminated culture, and in FIG. 3B, a cell 300 in a contaminated culture containing a dye deposit 360 that is indicative of mycoplasma contamination.

FIG. 4A shows RNA samples used in RT-PCR. The lanes are: a) 1 kB DNA Ladder, b) BHK-21 cells infected with Mycoplasma hyorhinis as a monolayer, c) the pellet of BHK-21 cells medium infected with Mycoplasma hyorhinis, and d) the pellet of Mycoplasma hyorhinis from mycoplasma enrichment broth (not from tissue culture cells).

FIG. 4B is a graphical representation of the expression of several Mycoplasma hyorhinis genes under conditions A-J: A) lon, 3T3 cells growing as a monolayer, B) lon, BHK-21 cells growing in DMEM, C) map, 3T3 cells growing as a monolayer, D) map, BHK-21 cells growing in DMEM, E) pepA, 3T3 cells growing as a monolayer, F) pepA, BHK-21 cells growing in DMEM, G) trxB, 3T3 cells growing as a monolayer, H) trxB, BHK-21 cells growing in DMEM, I) vacB, 3T3 cells growing as a monolayer, J) vacB, BHK-21 cells growing in DMEM.

FIG. 5 shows the results of testing of primers with genomic DNA from Mycoplasma hyorhinis. The PCR products were run on a 1.5% agarose gel. We performed 35 cycles of hot start PCR with an initial melt of 95° C. for 4 minutes, followed by a melt at 95° C. for 45 seconds, an annealing at 50° C. for 45 seconds, and a final extension step 72° C. for 7 minutes. The lane order includes a 1 kB DNA ladder (a), 5′-3′ exonuclease (b &k), gcp (c & l), lon (d & m), map (e & n), nfo (f & o), nox, (g&p), trxB (h &q), uvrA (i &r), p37 (j&s), all using 0.5 μl of DNA template (B-J) or 1 μl of DNA diluted 1:10.

FIG. 6 shows the results of testing of further PCR primers with genomic DNA from Mycoplasma hyorhinis. Lanes include 1 kB DNA ladder (a), gcp (b), hrcA (c), lgt (d), pepA (e), pepP pth (g), mc (h), uvrB (i), uvrC (j), vacB (k), 5′-3′ (1), nox (m), p37 (n). The PCR product for pepF amplified the correct size product as well (not shown).

FIG. 7 shows the results of testing by RT-PCR of all primers that worked under 50° C. AT. The RNA template used was the BHK-21 DMEM pellet that had been previously treated with DNase. For each 41 of a 1:10 dilution of template. RT, 45° C. for 10 minutes, 95° C. for 15 minutes, amp. cycled 35 times 95° C. 15 s 50° C. 45 s, final extension at 72° C. for 7 minutes. Lanes include 1 kB DNA ladder (a), 5′-3′(b), lgt (c), lon (d), map (e), nfo (f), nox (g), pepA (h), pepF (i), pth (j), rnc (k), trxB (1), uvrA (m), uvrB (n), vacB (O), p37 (p), no primer control.

FIG. 8 shows an agarose gel loaded with double stranded DNA (dsDNA, lanes b-i) or double stranded RNA (ribosomal RNA, lanes j-q) treated with M. hyorhinis extract from a cell culture infection or supernantants from infected or uninfected cell cultures. The respective lanes contain: a) 1 kb DNA ladder, b) dsDNA exposed to an aliquot of M. hyorhinis extract for 30 minutes, c) dsDNA exposed to an aliquot of the supernatant of an infected cell culture for 30 minutes, d) dsDNA exposed to an aliquot of the supernatant of an uninfected cell culture for 30 minutes, e) dsDNA exposed to H₂O for 30 minutes, f) dsDNA exposed to an aliquot of M. hyorhinis extract for 0 minutes, g) dsDNA exposed to an aliquot of the supernatant of an infected cell culture for 0 minutes, h) dsDNA exposed to an aliquot of the supernatant of an uninfected cell culture for 0 minutes, i) dsDNA exposed to H₂O for 0 minutes, j) ribosomal RNA exposed to an aliquot of M. hyorhinis extract for 30 minutes, k) ribosomal RNA exposed to an aliquot of the supernatant of an uninfected cell culture for 30 minutes, 1) ribosomal RNA exposed to an aliquot of the supernatant of an uninfected cell culture for 30 minutes, m) ribosomal RNA exposed to exposed to H₂O 30 minutes, n) ribosomal RNA exposed to an aliquot of M. hyorhinis extract for 0 minutes, o) ribosomal RNA exposed to an aliquot of the supernatant of an infected cell culture for 0 minutes, p) ribosomal RNA exposed to an aliquot of the supernatant of an uninfected cell culture for 0 minutes, and q) ribosomal RNA exposed to an aliquot of H₂O for 0 minutes.

FIG. 9 is a graph of the results of testing the thioredoxin reductase activities of M. hyorhinis, E. coli and S. aureus. There was no significant activity from 10⁴-10⁶ CFU/ml of S. aureus or E. coli using the DTNB substrate.

FIG. 10A and FIG. 10B are schematic diagrams of a substrate-linked enzymatic reporter reagent. In FIG. 10A, a bead 400 is covalently linked to horseradish peroxidase 420 by a molecule DTSSP 410 that is a substrate for a reductase such as trxB. In FIG. 10B, a bead 400 is covalently linked to luciferase 440 by a molecule Leu 430 that is a substrate for a protease such as pepA.

FIG. 11 is a graph showing the effect of 1 mM DTT in enhancing the fluorescence of trxB substrates 2′,7′-difluoro-4′-(2-(5-((dimethylaminophenyl) azo)pyridyl)dithiopropionyl aminomethyl)fluorescein (DFDMAP) and BODIPY®FL L-cystine.

FIG. 12 is a graph showing the effect of digitonin on the trxB assay.

FIG. 13 is a graph showing the effect of acid pH levels on the leu-MCA assay.

FIG. 14 is a graph showing the effect of basic pH levels on the leu-MCA assay.

FIG. 15 is a graph showing the sensitivity of the leu-MCA assay.

### DETAILED DESCRIPTION OF THE INVENTION

Many of the genomes of the genus Mycoplasma have been sequenced. It is apparent that the microorganism has few biosynthetic genes, and the microorganism can only thrive in very rich growth mediums. Using a genomic approach in which we compared the genomes of 10 different mycoplasma (M. gallisepticum, M. capricolum, M. genitalium, M. hyoppneumonia, M. mobile, M. mycoides, M. penetrans, M. pneumonia, M. pulmonis, and M. synoviae) at 40% sequence identity, we have identified 243 genes that are conserved in all Mycoplasma species studied to date. Mycoplasma species use an array of hydrolytic enzymes to uptake materials that compensates for having very few internal biosynthetic processes. The identified genes are listed in Table 1, below, using the M. capricolum notation.

The common genes included a variety of enzymes that can be grouped into seven classes: synthetic enzymes, hydrolytic enzymes, chaperones, permeases, kinases, transcription factors, and ribosomal proteins. The presence of acetate kinase has been disclosed as an assay for the presence of Mycoplasma (U.S. published patent application No. 2004/0265942). However, this assay is an enzyme cascade assay requiring luciferase and is not amenable to a simple and direct method for measuring contamination in culture and in vivo.

The hydrolytic enzymes are interesting targets both for diagnosis and the treatment of a mycoplasma infection because they are secreted and likely involved in infection and virulence. The common hydrolytic enzymes of Mycoplasma species include: proteases, such as the gene products of MCAP_{—}0157, MCAP_{—}0195, MCAP_{—}0267, MCAP_{—}0341, MCAP_{—}0509, MCAP_{—}0675, nucleases, such as the gene products of MCAP_{—}0047, MCAP_{—}0060, MCAP_{—}0097, MCAP_{—}0252, MCAP_{—}0492, MCAP_{—}0768, MCAP_{—}0773, MCAP_{—}0774, and reductases, such as the gene products of MCAP_{—}0101, MCAP_{—}0427, MCAP_{—}0779, and MCAP_{—}0858.

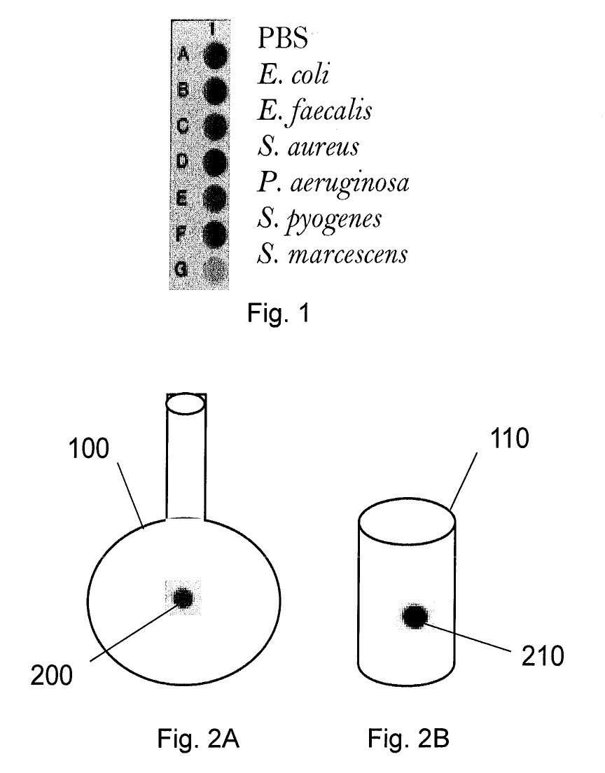

Reductase activity can be measured through a Azo dye that gets decolorized by the release of reductases from many bacterial cells. An azo dye such as reactive black 5 or DABCYL (4-((4-(dimethylamino)phenyl)azo)benzoic acid) is completely decolorized by many bacterial cultured supernatant after just 18 hours on incubation. A sensor placed on the bottom of a culture dish, buffer container or even on a swab for measuring the presence of mycoplasma in human fluids can be used to ascertain bacterial contamination or infection. The benefit of a simple azo dye sensor is low cost although it may not specifically detect different bacteria. FIG. 1 is a photograph of a microtiter plate containing reactive black 5 decolorized by incubation with different pathogenic bacteria. Each well was incubated with 10 μg of reactive black 5 plus 190 μl of filtered culture supernatant from the following bacterium: E. coli, E. faecalis, S. aureus, P. aeruginosa, S. pyogenes, and S. marcescens. Such azo dyes are decolorized by most bacteria after incubation with the dye for about 18 hours. The decolorization is indicative of reductases produced by the bacteria. Mycoplasma reductases such as the gene products of MCAP_{—}0101. MCAP_{—}0427, MCAP_{—}0779, and MCAP_{—}0858 can also decolorize such substrates.

In other embodiments, substrates for reductases are reagents that produce a fluorescent signal. Suitable such reagents include DTNB (5,5′-Dithio-bis-(2-nitrobenzoic acid), also known as Ellman's reagent.

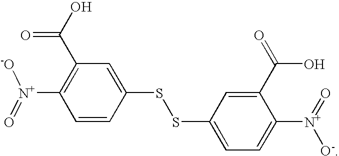

Two other suitable fluorogenic compounds are BODIPY®FL L-cystine,

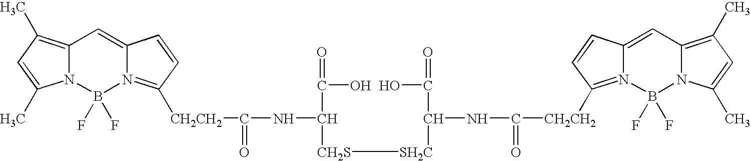

and 2′,7′-difluoro-4′-(2-(5-((dimethylaminophenyl)azo)pyridyl) dithiopropionyl aminomethyl)fluorescein,

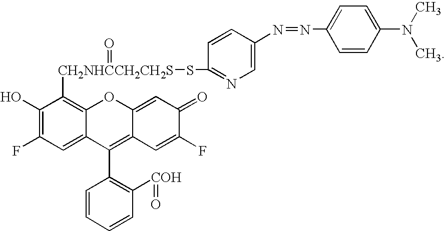

Specific peptidase substrates can be used to identify a specific bacterium. Published patent applications disclosing both specific and broad-spectrum targets for detection of pathogens include WO 2005/042770, WO2005/012556 and WO2004/087942, which are incorporated herein by reference. Mycoplasmas secrete a lysine-specific endopeptidase, an aminopeptidase and a carboxypeptidase that make it possible to specifically detect the presence of mycoplasma by using a substrate that is specific for these enzymes. Suitable aminopeptidases and carboxypeptidases have been purified by Watanabe and colleagues (Watanabe, T. (1988), Proteolytic activities of Mycoplasma salivarium, Adv Dent Res 2(2):297-300; Watanabe, T (1985) Proteolytic activity of mycoplasmas and ureaplasmas isolated freshly from human saliva, Medical Microbiology and Immunology 173(5): 251-255; Watanabe, T. et al., (1984) Aminopeptidase and caseinolytic activities of Mycoplasma salivarium Medical Microbiology and Immunology, 172 (4): 257-264). In a preferred embodiment, these purified or partially purified enzymes are used in a high-throughput screen to identify potential novel substrates.

Mycoplasmas produce both secreted and membrane-bound nucleases that are involved in obtaining nucleotides for DNA synthesis. See Minion, C. J. D. Goguen (1986) Identification and Preliminary Characterization of External Membrane-Bound Nuclease Activities in Mycoplasma pulmonis, Infection And Immunity, 51(1):352-354; Kannan, T. R.,& Baseman, J. B., (2006) ADP-ribosylating and vacuolating cytotoxin of Mycoplasma pneumoniae represents unique virulence determinant among bacterial pathogens. PNAS, 103:6724-6729; Bendjennat, M., et al., (1997) Purification and Characterization of Mycoplasma penetrans Ca2+/Mg2+-Dependent Endonuclease, Journal of Bacteriology 179:2210-2220; Minion, C. F., et al., (1993) Membrane-Associated Nuclease Activities in Mycoplasmas. Journal of Bacteriology 175:7842-7847.

RNA or DNA sequences that are efficiently hydrolyzed by Mycoplasma nucleases that labeled with a detectable colorimetric or fluorescent dye can be used to detect the presence of mycoplasma contamination. A dye such as blue dye number 1 is not decolorized by microorganisms and would be a good choice for a colorimetric reporter. The dye is labeled with a reactive aminoallyl-dUTP via a Klenow reaction using techniques known to one skilled in the art to covalently attach the dye to a nucleic acid. See Hasseman, J. J., et al., 2006 Microbial Genomic DNA Aminoallyl Labeling For Microarrays, The Institute For Genomic Research Standard Operating Procedure # M009. The aminoallyl groups on the nucleic acid would then be available for labeling with a reactive fluorescent or chromogenic dye molecule. The dye-labeled nucleic acid can be attached to the surface of a sterile bottle. If the bottle after opening became contaminated with mycoplasmas, the spot of color on the inner surface of the bottle would be released, indicating that the bottle is contaminated. FIG. 2A is a diagrammatic illustration of a contamination biosensor 200 placed on a container 100 for a reagent or culture medium. FIG. 2B a diagrammatic illustration of a contamination biosensor 210 placed on a container 110 for tissue culture.

Azo dyes such as reactive black 5 and DABCYL are decolorized by bacteria and can be used as a broad spectrum sensor for microbial contamination. Blue dye number 1, which is not decolorized by bacteria, can be used as a label of nucleic acids or a peptide to give a specific probe for mycoplasmas or other contaminating microorganism. Fluorescent probes or the release of fluorescent micro-spheres can be used to indicate contamination. Contamination can be measured by eye, using a fluorimeter or colorimeter or on a microscope stage.

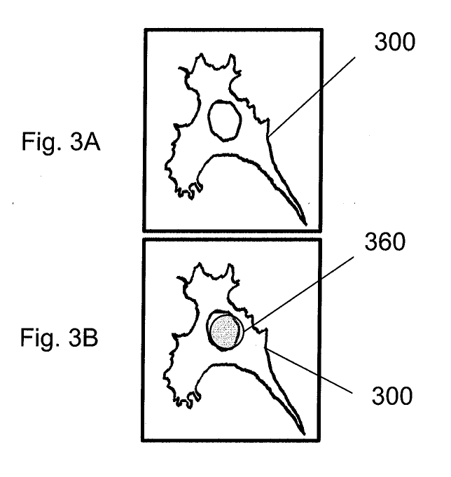

In another embodiment, a peptide or nucleic acid can be labeled with an acetoxymethyl ester of a dye, such as blue dye number 1, that upon hydrolytic cleavage would be taken up by cells in culture and thereby turn them blue to indicate the presence of mycoplasmas in the culture medium. FIG. 3 is a diagrammatic illustration of an embodiment of such a mycoplasmas contamination detection system for cell culture, showing in FIG. 3A a cell 300 in an uncontaminated culture, and in FIG. 3B, a cell 300 in a contaminated culture containing a dye deposit 360 that is indicative of mycoplasmas contamination. An acetoxymethyl ester derivative of blue dye number 1 coupled to a peptide or nucleic acid carrier would be impermeable to tissue culture cells until contamination with mycoplasmas. The proteases or nucleases from Mycoplasma spp. would cleave the carrier from the acetoxymethyl ester derivative of blue dye number 1, thereby allowing the acetoxymethyl ester derivative of blue dye number 1 to be taken up by the tissue culture cells. Tissue culture cells that become colored blue indicate that the culture was contaminated with mycoplasmas. The colored cells can be observed with a light microscope. Alternatively a cell permeable fluorescent dye can be used and the fluorescing cells can be detected with a fluorescence microscope.

TABLE 1
Genes characterized by sequences common to Mycoplasma spp. (40% identity)

| Gene | Symbol | Common Name |
|---|---|---|
| MCAP_0001 | dnaA | chromosomal replication initiator protein DnaA |
| MCAP_0002 | dnaN | DNA polymerase III, beta subunit |
| MCAP_0004 | ksgA | dimethyladenosine transferase |
| MCAP_0008 | dnaX | DNA polymerase III gamma-tau subunits |
| MCAP_0010 | tmk | thymidylate kinase |
| MCAP_0011 | | DNA polymerase III, delta prime subunit |
| MCAP_0017 | ftsH | ATP-dependent metalloprotease FtsH |
| MCAP_0022 | | acyl carrier protein phosphodiesterase, putative |
| MCAP_0026 | rpsR | 30S ribosomal protein S18 |
| MCAP_0035 | metG | methionyl-tRNA synthetase |
| MCAP_0038 | | ABC transporter, permease protein |
| MCAP_0039 | | ABC transporter, permease protein |
| MCAP_0040 | gyrA | DNA gyrase, A subunit |
| MCAP_0041 | gyrB | DNA gyrase, B subunit |
| MCAP_0045 | secA | preprotein translocase, SecA subunit |
| MCAP_0047 | | 5-3 exonuclease family protein |
| MCAP_0060 | | endonuclease IV |
| MCAP_0065 | rplK | 50S ribosomal protein L11 |
| MCAP_0066 | rplA | ribosomal protein L1 |
| MCAP_0067 | rplJ | 50S ribosomal protein L10 |
| MCAP_0068 | rplL | 50S ribosomal protein L7/L12 |
| MCAP_0070 | rpoB | DNA-directed RNA polymerase, beta subunit |
| MCAP_0071 | rpoC | DNA-directed RNA polymerase beta subunit |
| MCAP_0074 | | ribose 5-phosphate isomerase B, putative |
| MCAP_0075 | glyA | serine hydroxymethyltransferase |
| MCAP_0076 | upp | uracil phosphoribosyltransferase |
| MCAP_0078 | atpB | ATP synthase F0, subunit A |
| MCAP_0079 | atpE | ATP synthase F0, subunit c |
| MCAP_0082 | atpA1 | ATP synthase F1, alpha subunit |
| MCAP_0083 | atpG | ATP synthase F1, gamma subunit |
| MCAP_0084 | atpD1 | ATP synthase F1, beta subunit |
| MCAP_0094 | ptsG | PTS system, glucose-specific IIABC component |
| MCAP_0096 | smpB | SsrA-binding protein |
| MCAP_0097 | | Rnase R (VacB) and RNase II family 3-5 exoribonucleases |
| MCAP_0101 | nrdE | ribonucleoside-diphosphate reductase 2, alpha subunit |
| MCAP_0104 | prs | phosphoribosylpyrophosphate synthase |
| MCAP_0105 | pth | peptidyl-tRNA hydrolase |
| MCAP_0107 | dnaC | replicative DNA helicase |
| MCAP_0110 | cysS | cysteinyl-tRNA synthetase |
| MCAP_0111 | | RNA methyltransferase, TrmH family, group 3 |
| MCAP_0114 | nusG | transcription antitermination protein NusG |
| MCAP_0119 | | oligopeptide ABC transporter, ATP-binding protein |
| MCAP_0120 | | oligopeptide ABC transporter, ATP-binding protein |
| MCAP_0124 | | hydrolase, TatD family |
| MCAP_0130 | gltX | glutamyl-tRNA synthetase |
| MCAP_0136 | fba | fructose-1,6-bisphosphate aldolase, class II |
| MCAP_0140 | rpmE | ribosomal protein L31 |
| MCAP_0142 | | DHH phosphoesterase family protein, putative |
| MCAP_0143 | tdk | thymidine kinase |
| MCAP_0144 | prfA | peptide chain release factor 1 |
| MCAP_0145 | | modification methylase, HemK family |
| MCAP_0151 | rpsL | 30S ribosomal protein S12 |
| MCAP_0152 | rpsG | 30S ribosomal protein S7 |
| MCAP_0153 | fusA | translation elongation factor G |
| MCAP_0154 | tuf | translation elongation factor Tu |
| MCAP_0157 | pepA1 | cytosol aminopeptidase |
| MCAP_0159 | alaS | alanyl-tRNA synthetase |
| MCAP_0163 | | oligopeptide ABC transporter, ATP-binding protein |
| MCAP_0195 | pepA2 | cytosol aminopeptidase |
| MCAP_0200 | | spermidine/putrescine ABC transporter, permease protein |
| | | and spermidine/putrescine-binding protein |
| MCAP_0201 | | spermidine/putrescine ABC transporter, permease protein |
| MCAP_0203 | rplT | 50S ribosomal protein L20 |
| MCAP_0205 | infC | translation initiation factor IF-3 |
| MCAP_0208 | gmk | guanylate kinase |
| MCAP_0213 | eno | enolase4.2.1.11 |
| MCAP_0216 | hpt1 | hypoxanthine phosphoribosyltransferase |
| MCAP_0220 | pfkA | Phosphofructokinase |
| MCAP_0221 | pyk | pyruvate kinase |
| MCAP_0222 | thrS | threonyl-tRNA synthetase |
| MCAP_0223 | | NADH oxidase |
| MCAP_0224 | | lipoate-protein ligase |
| MCAP_0225 | pdhA | pyruvate dehydrogenase complex, El component, |
| | | alpha subunit |
| MCAP_0226 | pdhB | pyruvate dehydrogenase complex, E1 component, |
| | | beta subunit |
| MCAP_0228 | pdhD | dihydrolipoamide dehydrogenase |
| MCAP_0229 | pta | phosphate acetyltransferase |
| MCAP_0230 | ackA | acetate kinase |
| MCAP_0233 | ptsI | phosphoenolpyruvate-protein phosphotransferase |
| MCAP_0234 | crr | PTS system, glucose-specific IIA component |
| MCAP_0237 | rpsD | 30S ribosomal protein S4 |
| MCAP_0245 | | GTP-binding conserved hypothetical protein |
| MCAP_0251 | greA | transcription elongation factor GreA |
| MCAP_0252 | uvrC | excinuclease ABC, C subunit |
| MCAP_0255 | | conserved hypothetical protein |
| MCAP_0258 | valS | valyl-tRNA synthetase |
| MCAP_0260 | rpe | ribulose-phosphate 3-epimerase |
| MCAP_0261 | rsgA | ribosome small subunit-dependent GTPase A |
| MCAP_0267 | | metalloendopeptidase, putative |
| MCAP_0318 | proS | prolyl-tRNA synthetase |
| MCAP_0321 | lepA | GTP-binding protein LepA |
| MCAP_0323 | aspS | aspartyl-tRNA synthetase |
| MCAP_0324 | hisS | His-tRNA synthetase 6.1.1.21 |
| MCAP_0330 | rpsO | 30S ribosomal protein S15 |
| MCAP_0333 | infB | translation initiation factor IF-2 |
| MCAP_0336 | | transcription elongation protein nusA, putative |
| MCAP_0339 | polC | DNA polymerase III, alpha subunit, Gram-positive type |
| MCAP_0340 | cdsA | phosphatidate cytidylyltransferase |
| MCAP_0341 | | Xaa-Pro peptidase |
| MCAP_0342 | trpS | tryptophanyl-tRNA synthetase |
| MCAP_0358 | atpA2 | ATP synthase F1, alpha subunit |
| MCAP_0359 | atpD2 | ATP synthase F1, beta subunit |
| MCAP_0364 | | RNA methyltransferase, TrmH family |
| MCAP_0365 | | hydrolase of the HAD superfamily, putative |
| MCAP_0367 | hrcA | heat-inducible transcription repressor HrcA |
| MCAP_0369 | dnaK | Chaperone protein dnaK (Heat shock protein 70) |
| | | (Heat shock 70 kDaprotein) (HSP70) |
| MCAP_0371 | rpsB | 30S ribosomal protein S2 |
| MCAP_0372 | tsf | translation elongation factor Ts |
| MCAP_0374 | pyrH | uridylate kinase |
| MCAP_0375 | frr | ribosome recycling factor |
| MCAP_0376 | argS | arginyl-tRNA synthetase |
| MCAP_0383 | pheS | phenylalanyl-tRNA synthetase, alpha subunit |
| MCAP_0384 | pheT | phenylalanyl-tRNA synthetase, beta subunit |
| MCAP_0388 | mraW | S-adenosyl-methyltransferase MraW 2.1.1.- 479149 |
| MCAP_0393 | ileS | isoleucyl-tRNA synthetase |
| MCAP_0395 | | ribosomal large subunit pseudouridine synthase, RluA family |
| MCAP_0410 | | conserved hypothetical protein, TIGR00096 |
| MCAP_0412 | rplU | 50 ribosomal protein L21 |
| MCAP_0414 | rpmA | 50S ribosomal protein L27 |
| MCAP_0423 | | ImpB/MucB/SamB family protein |
| MCAP_0427 | | pyridine nucleotide-disulphide oxidoreductase |
| MCAP_0439 | ldh | L-lactate/malate dehydrogenase |
| MCAP_0445 | | triacylglycerol lipase |
| MCAP_0446 | | triacylglycerol lipase, putative |
| MCAP_0449 | | lipoate-protein ligase |
| MCAP_0454 | gtsA | glycerol ABC transporter, ATP-binding protein |
| MCAP_0456 | parC | DNA topoisomerase IV, A subunit |
| MCAP_0457 | parE | DNA topoisomerase IV, B subunit |
| MCAP_0462 | | RNA methyltransferase, TrmH family |
| MCAP_0465 | pgi | glucose-6-phosphate isomerase |
| MCAP_0469 | | aminotransferase, class V |
| MCAP_0472 | | HIT family protein |
| MCAP_0474 | ung | uracil-DNA glycosylase |
| MCAP_0476 | gid | glucose inhibited division protein |
| MCAP_0478 | metK | S-adenosylmethionine synthetase |
| MCAP_0479 | | conserved hypothetical protein TIGR00282 |
| MCAP_0481 | ftsY | signal recognition particle-docking protein FtsY |
| MCAP_0488 | rpmB | 50S ribosomal protein L28 |
| MCAP_0492 | | ribonuclease III |
| MCAP_0495 | | structural maintenance of chromosomes |
| | | (SMC) superfamily protein |
| MCAP_0497 | apt | adenine phosphoribosyltransferase |
| MCAP_0503 | rpoD | RNA polymerase sigma factor RpoD |
| MCAP_0504 | dnaG | DNA primase |
| MCAP_0505 | glyS | glycyl-tRNA synthetase |
| MCAP_0507 | era | GTP-binding protein Era |
| MCAP_0509 | | Peptidase C39 family protein |
| MCAP_0510 | | DJ-1 family protein |
| MCAP_0516 | lon | ATP-dependent protease La |
| MCAP_0517 | tig | trigger factor |
| MCAP_0519 | efp | translation elongation factor P |
| MCAP_0521 | | conserved hypothetical protein |
| MCAP_0523 | trmU | tRNA (5-methylaminomethyl-2-thiouridylate)- |
| | | methyltransferase |
| MCAP_0529 | | nicotinate (nicotinamide) nucleotide adenylyltransferase/ |
| | | conserved hypothetical domain |
| MCAP_0532 | | Spo0B-associated GTP-binding protein, putative |
| MCAP_0544 | rplS | 50S ribosomal protein L19 |
| MCAP_0545 | trmD | tRNA(guanine-N1)-methyltransferase |
| MCAP_0547 | rpsP | 30S ribosomal protein S16 |
| MCAP_0549 | ffh | signal recognition particle protein |
| MCAP_0551 | recA | recA protein |
| MCAP_0577 | engA | GTP-binding protein engA |
| MCAP_0578 | cmk | cytidylate kinase |
| MCAP_0581 | ppa | inorganic pyrophosphatase |
| MCAP_0589 | | ribulose-phosphate 3-epimerase, putative |
| MCAP_0601 | scpB | chromosomal segregation and condensation protein B |
| MCAP_0606 | | hydrolase, alpha/beta fold family |
| MCAP_0609 | | Uncharacterised membrane protein, UPF0154 family |
| MCAP_0610 | tkt | transketolase |
| MCAP_0613 | | glucose-inhibited division protein, putative |
| MCAP_0616 | | fructose/tagatose bisphosphate aldolase, class II |
| MCAP_0623 | | metallo-beta-lactamase superfamily protein |
| MCAP_0631 | pgk | phosphoglycerate kinase |
| MCAP_0632 | gap | glyceraldehyde-3-phosphate dehydrogenase |
| MCAP_0635 | mutM | formamidopyrimidine-DNA glycosylase |
| MCAP_0636 | polA | DNA polymerase I |
| MCAP_0637 | dnaE | DNA-directed DNA polymerase III (polc) |
| MCAP_0639 | tyrS | tyrosyl-tRNA synthetase |
| MCAP_0659 | leuS | leucyl-tRNA synthetase |
| MCAP_0662 | rpsI | 30S ribosomal protein S9 |
| MCAP_0663 | rplM | 50S ribosomal protein L13 |
| MCAP_0666 | | cobalt ABC transporter, permease protein |
| MCAP_0667 | | cobalt ABC transporter, ATP-binding protein, putative |
| MCAP_0668 | | cobalt ABC transporter, ATP-binding protein, putative |
| MCAP_0669 | rplQ | 50S ribosomal protein L17 |
| MCAP_0670 | rpoA | DNA-directed RNA polymerase, alpha chain |
| MCAP_0671 | rpsK | 30S ribosomal protein S11 |
| MCAP_0672 | rpsM | 30S ribosomal protein S13 |
| MCAP_0675 | map | methionine aminopeptidase, type I |
| MCAP_0676 | adk | adenylate kinase |
| MCAP_0677 | secY | preprotein translocase, SecY subunit |
| MCAP_0678 | rplO | 50S ribosomal protein L15 |
| MCAP_0679 | rpsE | 30S ribosomal protein S5 |
| MCAP_0680 | rplR | 50S ribosomal protein L18 |
| MCAP_0681 | rplF | 50S ribosomal protein L6 |
| MCAP_0682 | rpsH | ribosomal protein S8 |
| MCAP_0684 | rplE | ribosomal protein L5 |
| MCAP_0686 | rplN | ribosomal protein L14 |
| MCAP_0687 | rpsQ | ribosomal protein S17 |
| MCAP_0689 | rplP | ribosomal protein L16 |
| MCAP_0690 | rpsC | ribosomal protein S3 |
| MCAP_0691 | rplV | ribosomal protein L22 |
| MCAP_0692 | rpsS | 30S ribosomal protein S19 |
| MCAP_0693 | rplB | 50S ribosomal protein L2 |
| MCAP_0694 | rplW | 50S ribosomal protein L23 |
| MCAP_0695 | rplD | 50S ribosomal protein L4 |
| MCAP_0696 | rplC | 50 ribosomal protein L3 |
| MCAP_0697 | rpsJ | 30S ribosomal protein S10 |
| MCAP_0707 | | potassium uptake protein, TrkH family, putative |
| MCAP_0708 | | potassium uptake protein, TrkA family, putative |
| MCAP_0709 | gatB | glutamyl-tRNA(Gln) amidotransferase, B subunit |
| MCAP_0710 | gatA | glutamyl-tRNA(Gln) amidotransferase, A subunit |
| MCAP_0712 | ligA | DNA ligase, NAD-dependent |
| MCAP_0714 | | ribosomal large subunit pseudouridine synthase, RluA family |
| MCAP_0716 | ptsH | phosphocarrier protein hpr |
| MCAP_0750 | tpiA | triosephosphate isomerase |
| MCAP_0751 | | HAD-superfamily hydrolase subfamily IIB, protein |
| MCAP_0752 | gpmI | 2,3-bisphosphoglycerate-independent |
| | | phosphoglycerate mutase |
| MCAP_0755 | deoC | deoxyribose-phosphate aldolase |
| MCAP_0757 | deoA | pyrimidine-nucleoside phosphorylase |
| MCAP_0761 | trmB | tRNA (guanine-N(7)-)-methyltransferase |
| MCAP_0765 | hpt2 | hypoxanthine phosphoribosyltransferase |
| MCAP_0768 | | deoxyribonuclease, TatD family |
| MCAP_0773 | uvrB | excinuclease ABC, B subunit |
| MCAP_0774 | uvrA | excinuclease ABC, A subunit |
| MCAP_0778 | lgt1 | prolipoprotein diacylglyceryl transferase |
| MCAP_0779 | trxB | thioredoxin reductase |
| MCAP_0780 | lgt2 | prolipoprotein diacylglyceryl transferase |
| MCAP_0781 | | conserved hypothetical protein |
| MCAP_0792 | topA | DNA topoisomerase I |
| MCAP_0805 | engD | GTP-dependent nucleic acid-binding protein engD |
| MCAP_0807 | gidB | methyltransferase gidB |
| MCAP_0808 | pgsA | CDP-diacylglycerol--glycerol-3-phosphate 3- |
| | | phosphatidyltransferase |
| MCAP_0814 | | transporter protein, putative |
| MCAP_0818 | rpsT | 30S ribosomal protein S20 |
| MCAP_0819 | trmE | tRNA modification GTPase TrmE |
| MCAP_0821 | | glycoprotease family protein |
| MCAP_0824 | asnS | asparaginyl-tRNA synthetase |
| MCAP_0834 | | hydrolase, haloacid dehalogenase-like family, putative |
| MCAP_0836 | lysS | lysyl-tRNA synthetase |
| MCAP_0839 | serS | seryl-tRNA synthetase |
| MCAP_0844 | | PTS system glucose-specific IIBC component |
| MCAP_0849 | deoD | purine nucleoside phosphorylase |
| MCAP_0856 | gidA | glucose inhibited division protein A |
| MCAP_0858 | | pyridine nucleotide-disulphide oxidoreductase |
| MCAP_0860 | | membrane protein, putative |
| MCAP_0870 | rpmH | 50S ribosomal protein L34 |

### Example 1

### Mycoplasma hyorhinis Enzymes

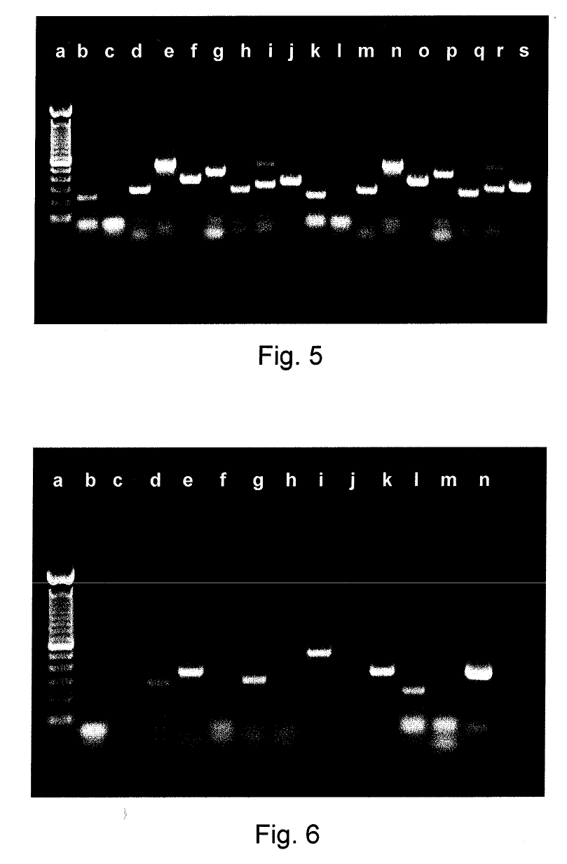

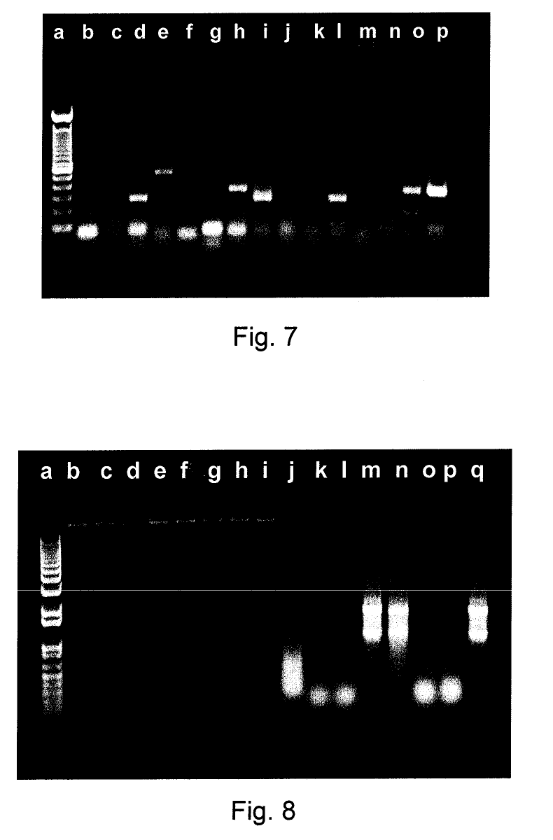

The subset of Mycoplasma genes from the Mycoplasma hyorhinis genome that were selected for further study are listed in Table 2, below. DNA PCR primers were made for each of these genes and the PCR are shown in FIG. 5 and FIG. 6.

TABLE 2
Hydrolytic enzymes in common among Mycoplasma ssp.

| | Gene | Symbol | Common Name |
|---|---|---|---|
| 1 | MHP7448_0010 | hrcA | heat-inducible transcription repressor |
| 2 | MHP7448_0037 | vacB | VACB-like ribonuclease II |
| 3 | MHP7448_0062 | nfo | endonuclease IV |
| 4 | MHP7448_0066 | uvrC | excinuclease ABC subunit C |
| 5 | MHP7448_0082 | nox | NADH oxidase |
| 6 | MHP7448_0091 | uvrA | excinuclease ABC subunit A |
| 7 | MHP7448_0097 | lgt | prolipoprotein diacylglyceryl transferase |
| 8 | MHP7448_0098 | trxB | thioredoxin reductase |
| 9 | MHP7448_0173 | map | methionine aminopeptidase |
| 10 | MPH7448_0204 | pth | prptidyl-tRNA hydrolase |
| 11 | MHP7448_0398 | rnc | ribonuclease III |
| 12 | MHP7448_0464 | pepA | leucyl aminopeptidase |
| 13 | MHP7448_0521 | pepF | oligoendopeptidase F |
| 14 | MHP7448_0524 | lon | heat shock ATP-dependent protease |
| 15 | MHP7448_0581 | | 5′-3′ exonuclease |
| 16 | MHP7448_0635 | gcp | O-sialoglycoprotein endopeptidase |
| 17 | MHP7448_0648 | uvrB | excinuclease ABC subunit B |
| 18 | MHP7448_0659 | pepP | XAA-PRO aminopeptidase |

### Purification of Total RNA

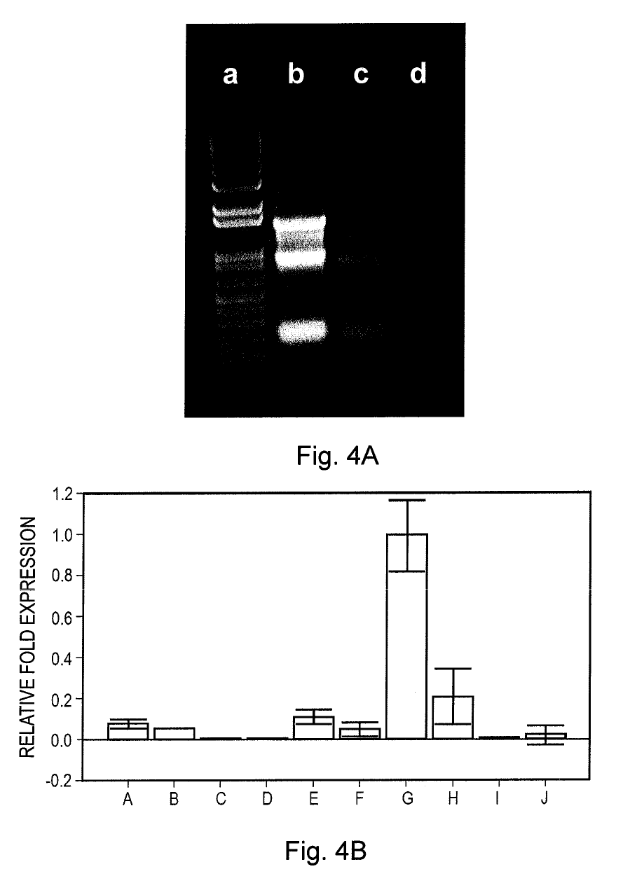

Total RNA was isolated from Mycoplasma hyorhinis grown in BHK-21 and Swiss 3T3 tissue culture cells (FIG. 4A). RNA was purified both from the infected tissue culture media and culture cells (BHK-21 & Swiss 3T3) from the infected dish. RNA was purified using either the Invitrogen Triazol® Max bacterial RNA isolation kit or an acid phenol-guanidium thiocyanate and chloroform extraction procedure. Although the acid phenol-guanidium thiocyanate and chloroform extraction procedure had better quality RNA as judged by gel electrophoresis in a 1.5% agarose gel, the RNA from the Triazol® Max bacterial RNA kit had more mycoplasma RNA as judged by PCR of the p37 control gene. As a positive control for the RT-PCR reactions, we amplified the p37 gene from the total infected BHK-21 and 3T3 cell media. As a negative control, we also isolated total RNA from uninfected tissue culture media and uninfected BHK-21 or 3T3 cells and then performed RT-PCR using the p37 primer set. RT PCR was performed in a iCycler iQ PCR Detection System (Bio-Rad) using the SYBR Green One-Step Quantitative RT-PCR kit. Alternatively, for the preliminary studies RT PCR was performed with the Ambion Ag-Path kit. FIG. 4A shows an exemplary result of agarose gel electrophoresis of the RNA samples used in RT-PCR. The lanes are: a) 1 kB DNA Ladder, b) BHK-21 cells infected with Mycoplasma hyorhinis as a monolayer, c) the pellet of BHK-21 cells medium infected with Mycoplasma hyorhinis, and d) the pellet of Mycoplasma hyorhinis from mycoplasma enrichment broth (not from tissue culture cells).

The results of preliminary studies indicate that the following genes vacB, trxB, map, pepA, lon, and uvrB are transcribed at a high level. FIG. 4B is a graphical representation of the expression of several Mycoplasma hyorhinis genes under conditions A-J: A) lon, 3T3 cells growing as a monolayer, B) lon, BHK-21 cells growing in DMEM, C) map, 3T3 cells growing as a monolayer, D) map, BHK-21 cells growing in DMEM, E) pepA, 3T3 cells growing as a monolayer, F) pepA, BHK-21 cells growing in DMEM, G) trxB, 3T3 cells growing as a monolayer, H) trxB, BHK-21 cells growing in DMEM, I) vacB, 3T3 cells growing as a monolayer, J) vacB, BHK-21 cells growing in DMEM. The level of expression from strongest to weakest for these abundant mRNA is trxB>pepA>map>vacB>lon>uvrB.

Substrates for the hydrolytic enzymes corresponding to these putative abundant mRNAs from Mycoplasma were identified using both literature and patent searches. Certain selected examples are provided in Table 3, below.

TABLE 3
Mycoplasma Hydrolase Substrates

| Gene Symbol | Substrates | References |
|---|---|---|
| vacB | ds RNAse activity | J. Cell. Physiol. 143(3) 416-419 |
| trxB | DTNB | Cayman Chemical Company |
| Map | Rhodamine based fluorogenic | J. Biomol. Screening 7(6) |
| | substrates | 531-540, 2002 |
| pepA | Leucine at N-terminal of a peptide | Curr. Microbiol. 48(1)32-38, 2004 |
| lon | Glutaryl-Ala-Ala-Phe- | J. Biol. Chem. 260(22) 1 1985 |
| | methoxynaphthylamine + ATP | |
| uvrB | (UvrA)2(UvrB)1 com | Proc. Natl Soc. USA 86(14) |
| | | 5237-5241, 1985. |

The genes that were determined by quantitative RT-PCR results to be highly expressed in Mycoplasma hyorhinis when infecting 3T3 cells or BHK-21 cells: trxB, pepA, lon, vacB, map, and uvrB. The substrates for the enzymes produced by these gene products is reported in Table 4, below.

TABLE 4
Mycoplasma hyorhinis
Gene Encoding Enzymes and Enzyme Substrates

| Gene | Enzyme | Substrate |
|---|---|---|
| trxB | Thioredoxin reductase | DTNB |
| pepA | Leucine aminopeptidase | leu-MCA |
| lon | ATP-dependent protease | glt-ala-ala-phe-MCA |
| map | Methionine aminopeptidase | met-MCA |
| vacB | exoribonuclease | dsRNA |
| uvrB | excinuclease | dsDNA |

We examined the nuclease activities of VacB and UvrB by challenging extracts from M. hyorhinis isolated from a cell culture infection or medium from Mycoplasma-infected or uninfected cell culture with either double stranded (ds) RNA or dsDNA. The reaction was incubated for 30 minutes at 37° C. and the products were then analyzed by agarose gel electrophoresis, as shown in FIG. 8.

FIG. 8 shows an agarose gel loaded with double stranded DNA (dsDNA, lanes b-i) or double stranded RNA (ribosomal RNA, lanes j-q) treated with M. hyorhinis extract from a cell culture infection or supernatants from infected or uninfected cell cultures. The respective lanes contain: a) 1 kb DNA ladder, b) dsDNA exposed to an aliquot of M. hyorhinis extract for 30 minutes, c) dsDNA exposed to an aliquot of the supernatant of an infected cell culture for 30 minutes, d) dsDNA exposed to an aliquot of the supernatant of an uninfected cell culture for 30 minutes, e) dsDNA exposed to H₂O for 30 minutes, f) dsDNA exposed to an aliquot of M. hyorhinis extract for 0 minutes, g) dsDNA exposed to an aliquot of the supernatant of an infected cell culture for 0 minutes, h) dsDNA exposed to an aliquot of the supernatant of an uninfected cell culture for 0 minutes, i) dsDNA exposed to H₂O for 0 minutes, j) ribosomal RNA exposed to an aliquot of M. hyorhinis extract for 30 minutes, k) ribosomal RNA exposed to an aliquot of the supernatant of an uninfected cell culture for 30 minutes, l) ribosomal RNA exposed to an aliquot of the supernatant of an uninfected cell culture for 30 minutes, m) ribosomal RNA exposed to exposed to H₂O 30 minutes, n) ribosomal RNA exposed to an aliquot of M. hyorhinis extract for 0 minutes, o) ribosomal RNA exposed to an aliquot of the supernatant of an infected cell culture for 0 minutes, p) ribosomal RNA exposed to an aliquot of the supernatant of an uninfected cell culture for 0 minutes, and q) ribosomal RNA exposed to an aliquot of H₂O for 0 minutes.

There was no detectable dsDNAse activity (lanes b & c) in the M. hyorhinis extract or M. hyorhinis-infected medium suggesting that there is insufficient urvB activity for this enzyme to serve as a suitable basis for a diagnostic test for Mycoplasma contamination. Although dsRNAse activity was observed in the M. hyorhinis extract and M. hyorhinis-infected medium (lanes j and k), the uninfected tissue culture media control also had appreciable dsRNAse activity. This finding indicates that vacB activity would not be a suitable basis for a Mycoplasma diagnostic test due to cross-reactivity from ribonucleases present in the culture medium of uninfected cells.

TABLE 5
Mycoplasma hyorhinis
Assays for Proteolytic Activity

| Gene | Enzyme | Substrate | Condition | Vmax |
|---|---|---|---|---|
| lon | ATP-dependent protease | glt-ala-ala-phe-MCA | Buffer | 55.8 |
| | | | Uninfected medium | 1460 |
| | | | Infected medium | 1802 |
| | | | Mycoplasma | 3354 |
| | | | positive control | |
| pepA | Leucine aminopeptidase | leu-MCA | Buffer^{A} | 2874 |
| | | | Uninfected medium | 454 |
| | | | Infected medium | 906 |
| | | | Mycoplasma | 522 |
| | | | positive control | |
| map | Methionine aminopeptidase | met-MCA | Buffer | 19.4 |
| | | | Uninfected medium | 430 |
| | | | Infected medium | 627 |
| | | | Mycoplasma | 733 |
| | | | positive control | |

^{A}This reading is probably artefactually high, and may indicate a bubble in the well. Subsequent experiments showed that Vmax in buffer was essentially zero.

Further studies examined the suitability of the aminopeptidases map or lon or the protease pepA for use in a diagnostic test. These studies used several fluorogenic substrates consisting of small chain amino acids coupled to a methoxy coumarin fluorescent probe (MCA, (7-methoxycoumarin-4-yl)acetyl). The results of the initial studies are provided in Table 5, above. The test conditions were “buffer,” phosphate-buffered saline (PBS), “uninfected medium,” medium from cell cultures not infected with Mycoplasma, “infected medium,” medium from cell cultures infected with Mycoplasma, and a Mycoplasma positive control derived from a Mycoplasma culture. In each case the uninfected media control had substantial background proteolytic activity, with pepA being the most candidate with a signal-to-noise ratio (S/N) of about 2, where S/N=(Vmax infected medium)/(Vmax uninfected medium).

### Example 2

### Detection of Mycoplasma Using trxB

In contrast to the protease and the double stranded nuclease markers, thioredoxin reductase (trxB) had significant activity specific to tissue culture cells co-infected with Mycoplasma hyorhinis. In earlier studies, we demonstrated that Mycoplasma thioredoxin reductase activity was measured in infected culture medium using the substrate DTNB (5,5′-Dithio-bis-(2-nitrobenzoic acid), also known as Ellman's reagent.

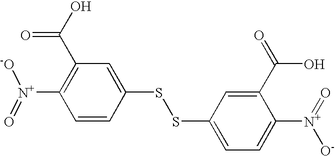

Other suitable fluorogenic thioredoxin reductase substrates have been reported in the literature.

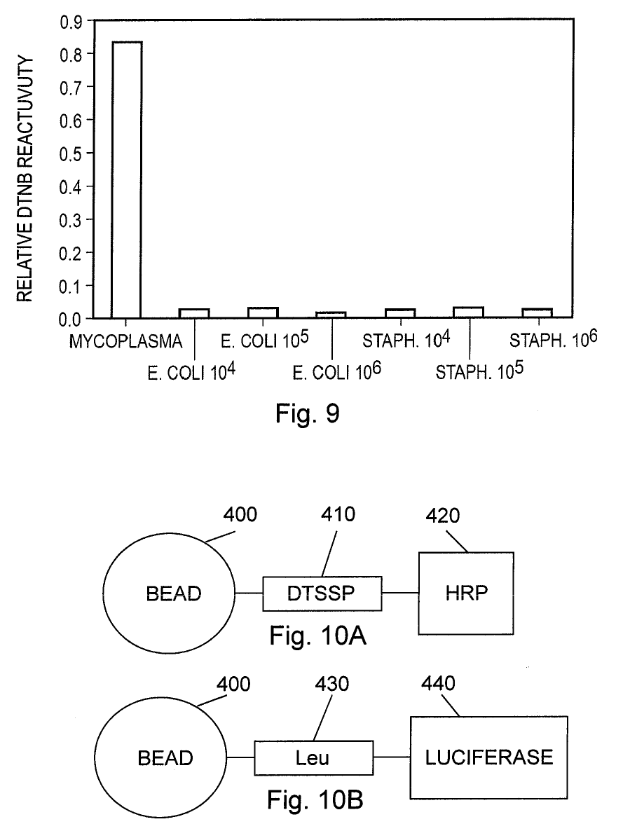

Since thioredoxin reductases are widely distributed in eukaryotes and prokaryotic cells, there is a possibility that thioredoxin reductases from other microbes may cross-react with this assay to give a false positive result. One possible approach would be to use gentle lysis buffers that disrupt Mycoplasma cells, which do not have a cell wall, but do not appreciably lyse other bacteria that possess a cell wall. Studies demonstrated that there was no appreciable hydrolysis of DTNB by up to 10⁶ CFU/ml E. coli or S. aureus (FIG. 9). This result indicates that the thioredoxin reductases of other gram-positive and gram-negative bacteria, represented by E. coli and S. aureus, are either minor enzymatic components or have a much lower specific activity than that of M. hyorhinis. This finding is consistent with the previous demonstration that Mollicutes such as Mycoplasma have a very highly active thioredoxin reductase system (NTS) (0.09-0.25 SA units) in the presence of NADPH. This high NTS activity is presumed to be useful for Mycoplasma for the detoxification of reactive oxygen compounds, since the Mollicutes have simple genomes that lack the genes encoding enzymes such as catalase, peroxidase and oxygen dismutase that function to remove H₂O₂ and other oxygen radicals in other bacteria (Gibson, D. G., et al., Complete chemical synthesis, assembly, and cloning of a Mycoplasma genitalium genome. Science. 319(5867):1215-1220 (2008)).

Although initial attempts to detect trxB with DTNB were successful and the DTNB did not cross react with Staphylococcus aureus or Escherichia coli (FIG. 9), the sensitivity of the assay was unacceptable (>10⁶ CFU/ml). Other fluorescent probes such as BODIPY®FL L-cystine and the 2′,7′-difluoro-4′-(2-(5-((dimethyl amino phenyl)azo)pyridyl)dithiopropionyl aminomethyl)fluorescein (abbreviated as DFDMAP-fluorescein) were studied to improve the sensitivity and the signal-to-noise ratio of the assay. Assays were performed with 40 mM Tris pH 7.2, 100 mM NaCl±detergent.

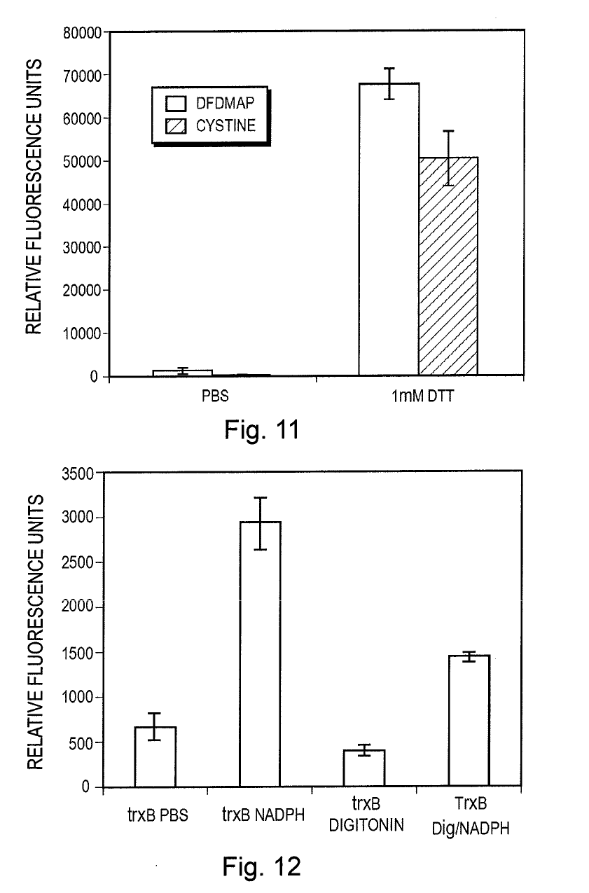

A detergent lysis buffer procedure that would hydrolyze the simple Mycoplasma cell membranes but not lyse the tissue culture cells was needed. Methyl-6-O—(N-heptylcarbamoyl)-α-D-glucopyranoside (HECAMEG) is a preferred detergent. We have found that 0.5% HECAMEG was sufficient to lyse Mycoplasma cells while not disrupting the membranes of the cells grown in the culture medium. In contrast, 0.25% Triton X-100°, 0.4% BriJ 35®, and digitonin resulted in either significant increase in the background or loss in the true level of trxB activity. The activity of the fluorescent substrates is low compared to reduction with DTT suggesting that they may not be ideal or specific for trxB (FIG. 11). As expected, 0.1 mM NADPH enhanced the activity of trxB (FIG. 12). As a substrate for trxB, Bodipy® FL L-cystine provided a signal that was 7500 times that of the buffer control, however this signal was an artifact of the digitonin. DFDMAP fluorescein had a moderately improved detection level of 10,000-20,000 RFU at 10⁷-10⁸ CFU/ml.

An alternative substrate to produce the thioredoxin reductase signal was to measure the release of horseradish peroxidase (HRP) from a chromatography bead tethered with the heterobifunctional crosslinking reagent 3,3′-dithiobis[sulfosuccinimidylpropionate] (DTSSP).

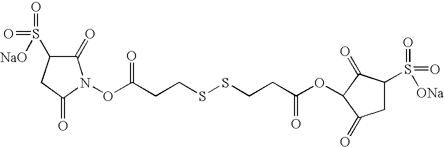

The HRP-DTSSP-BEAD conjugate is shown schematically in FIG. 10A.

It was expected that frxB would be able to reduce the disulfide bridge of DTSSP, thereby releasing the HRP to react with its substrate tetramethylbenzidine (TMB), and in the presence of Mycoplasma, produce a blue color. However, it was found that the HRP-DTSSP-BEAD conjugate also cross-reacted to the tissue culture uninfected medium sample.

Other alternative substrates can use fluorescence energy transfer (FRET) with a disulfide bridge between EDANS and DABSYL, as shown below:

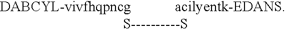

Alternatively, DFDMAP and BODIPY® FL L-cysteine moieties could be coupled to the thioredoxin peptide or the central Gly-Ala residues to enhance the specificity of these fluorescent probes. Initial studies of these approaches have not shown improved sensitivity or reduction of background of the uninfected media control.

### Example 3

### Detection of Mycoplasma Using Proteases

The proteases pepA, lon, and map were evaluated for use in the detection of Mycoplasma. pepA and lon were found to be expressed at a higher level than map based on RT-qPCR results (FIG. 5 and FIG. 6). Arginine amino peptidase activity has also been reported in Mycoplasma species.

A presently preferred substrate is leu-MCA that had significant activity above the uninfected media control under the gentle conditions used to lyse the Mycoplasma (0.05% HECAMEG, 1 mM MgCl₂, 100 mM NaCl, 40 mM Tris buffer, pH 8.5). The MCA-Leu substrate produces a signal level of 500 mOD in 30 minutes with M. hyorhinis, while M. hyorhinis has weak activity for arg-MCA. Results are provided in Table 6, below.

TABLE 6
Vmax Measured Using MCA Labeled Substrates
Under Different Conditions

| | Vmax | | | | |
|---|---|---|---|---|---|
| Condition | Arg-MCA | Leu-MCA | Met-MCA | Leu/Met | All 3 |
| Buffer | 0 | 236 | 125 | 0 | 193 |
| Medium | 676 | 2118 | 1731 | 2024 | 1443 |
| Uninfected | 29034 | 17422 | 19004 | 14937 | 25770 |
| Medium | | | | | |
| Infected | 60903 | 415190 | 302082 | 373456 | 271170 |
| Medium | | | | | |
| Signal/Noise | 2.10 | 23.83 | 15.90 | 25.00 | 10.52 |

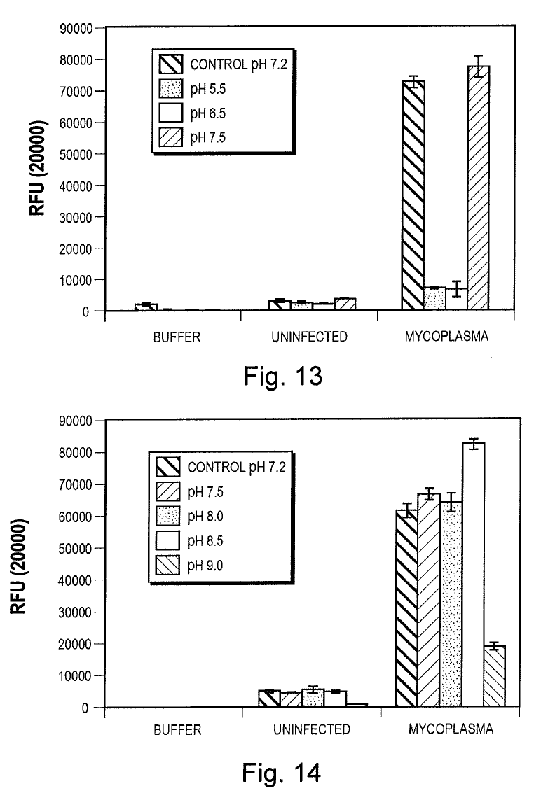

In further studies, we determined that the background of the uninfected cells could be reduced even further by adjusting the pH, with an optimum at pH 8.5. FIG. 13 is a graph showing the effect of acid pH levels on the leu-MCA assay. FIG. 14 is a graph showing the effect of basic pH levels on the leu-MCA assay.

Detergent lysis of M. hyorhinis with HECAMEG gives better signal than sonication. Manganese or magnesium also improves the signal to noise ratio. In the studies on the effects of divalent cations, 1 mM MgCl₂ of the standard mixture was replaced by 1 mM MnCl₂, 1 mM MgSO₄ or 1 mM EDTA, as indicated in Table 7, below.

TABLE 7
Effect of Divalent Cations
Vmax, MCA-Leu Substrate

| Condition | EDTA | MnCl₂ | MgSO₄ | MnCl₂ |
|---|---|---|---|---|
| Buffer | 0 | 0 | 483 | 84 |
| Medium | 1710 | 1703 | — | — |
| Uninfected | 3873 | 3819 | 5125 | 3032 |
| Infected | 45241 | 89467 | 90365 | 95120 |
| Signal/Noise | 11.68 | 23.43 | 17.63 | 31.37 |

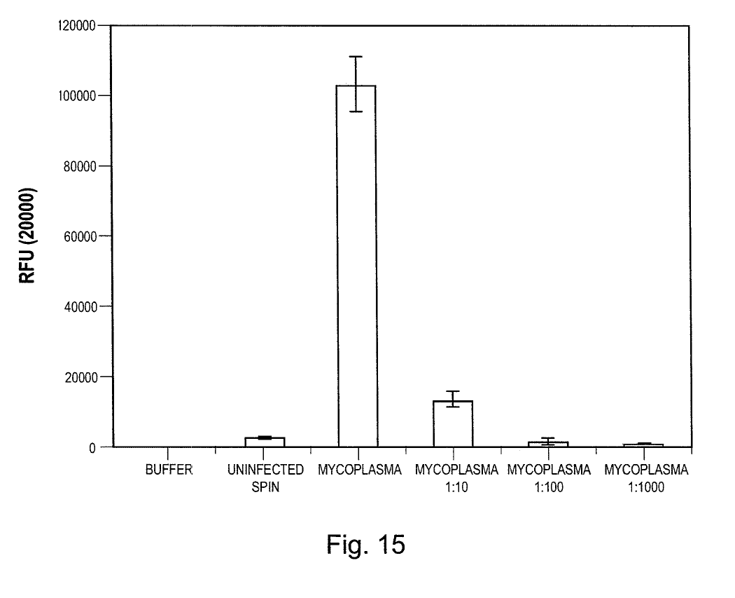

Using the leu-MCA substrate, a sensitivity of 10⁵ CFU/ml can be achieved (FIG. 15). Further increases in sensitivity may be obtained using a bis-leu rhodamine 110 labeled substrate, or using luciferase-leucine-bead complex, as shown schematically in FIG. 10B.

The sensitivity of the present assay using the leu-MCA substrate was compared to two commercially available Mycoplasma detection tests: a Mycoplasma PCR ELISA test (Roche cat #11 663 925 910), and the MycoAlert Sample Kit, (Lonza cat #LT37-618). The results are presented in Table 8, below.

TABLE 8
Test Sensitivity Comparison

| | Present leu-MCA | PCR ELISA | MycoAlert |
|---|---|---|---|
| | Assay | test | Sample Kit |
| M. hyorhinis | 10⁵ | 80 | 10⁶ |
| (CFU/ml) | | | |
| Time Required | 20 minutes | 2 Days | 20 minutes |
| For Test | | | |

The cross reactivity of the present assay using the leu-MCA substrate was evaluated with the following microorganisms: two bacteria (S. aureus, E. coli) and three species of fungus (Candida albicans, Aspergillis niger, Saccharomyces cerevisiae). Only in the case of a completely turbid cultures was there weak low cross reactivity with the present assay using the leu-MCA substrate.

What is claimed:

1. A biosensor for detecting the presence or absence of mycoplasma contamination comprising a support and a detectably labeled substrate for an hydrolytic enzyme produced and/or secreted by a mycoplasma, wherein the substrate is attached to a support.

2. The biosensor of claim 1 wherein the enzyme is selected from the group consisting of proteases, reductases and nucleases.

3. The biosensor of claim 1 wherein the enzyme is selected from the group consisting of the gene product of pepA1, MCAP_{—}0195, MCAP_{—}0267, MCAP_{—}0341, MCAP_{—}0509, MCAP_{—}0675 and mixtures thereof.

4. The biosensor of claim 1 wherein the enzyme is a reductase and the substrate is reactive black 5,5,5′-dithio-bis-(2-nitrobenzoic acid) (DTNB), BODIPY®FL L-cystine, or 2′,7′-difluoro-4′-(2-(5-((dimethyl amino phenyl)azo)pyridyl)dithiopropionyl aminomethyl)fluorescein (DFDMAP-fluorescein).

5. The biosensor of claim 4 wherein the reductase is selected from the group consisting of the gene product of nrdE (such as MCAP_{—}0101), MCAP_{—}0427, trxB (thioredoxin reductase, such as MCAP_{—}0779 or MHP7448_{—}0098), MCAP_{—}0858 and mixtures thereof.

6. The biosensor of claim 1 wherein the mycoplasma-specific enzyme is a nuclease and the detectable label is an acetoxymethyl ester or maleimide derivative of blue dye number 1 coupled to an aminollyl-dNTP labeled nucleic acid substrate of the mycoplasma-specific nuclease.

7. The biosensor of claim 6 wherein the nuclease is selected from the group consisting of the gene product of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof.

8. The biosensor of claim 1 wherein the support is a reagent container, a bead, a polymer film, a culture medium container or a cell culture container.

9. A method of detecting mycoplasma contamination of a cell culture comprising the steps of providing a cell-permeable detectable label coupled to a cell-impermeant carrier in the culture medium wherein cleavage of the detectable label by a mycoplasma-specific enzyme is followed by uptake of the detectable label into cells; and detecting labeled cells, thereby detecting mycoplasma contamination of the cell culture.

10. The method of claim 9 wherein the mycoplasma-specific enzyme is a protease and the detectable label is leucine-(7-methoxycoumarin-4-yl)acetyl (leu-MCA), arginine-(7-methoxycoumarin-4-yl)acetyl (arg-MCA), methionine-(7-methoxycoumarin-4-yl)acetyl (met-MCA), or an acetoxymethyl ester.

11. The method of claim 10 wherein the protease is selected from the group consisting of pepA1 (MCAP_{—}0157), pepA2 (MCAP_{—}0195), pepA (leucyl aminopeptidase, such as MHP7448_{—}0464), MCAP_{—}0267 (metalloendopeptidase), pepP (Xaa-Pro endopeptidase, such as MCAP_{—}0341 or MHP7448_{—}0649), MCAP_{—}0509, mapP (methionine amino peptidase, MCAP_{—}0675 or MHP7448_{—}0173), and mixtures thereof.

12. The method of claim 9 wherein the mycoplasma-specific enzyme is a nuclease and the detectable label is an acetoxymethyl ester of derivative of blue dye number 1 coupled to a nucleic acid substrate of the mycoplasma-specific nuclease

13. The method of claim 12 wherein the nuclease is selected from the group consisting of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof.

14. A method of determining the presence or absence of mycoplasma in a sample, comprising the steps of:
contacting the sample with a detectably labeled substrate for an enzyme produced and/or secreted by a mycoplasma under conditions that result in the modification of the substrate by the enzyme; and
detecting the modification or the absence of the modification of the substrate wherein modification of the substrate indicates the presence of mycoplasma in the sample, and wherein the absence of modification of the substrate indicates the absence of mycoplasma in the sample.

15. The method of claim 14 wherein the level of the detectable label is quantitatively related to the presence or amount of mycoplasma in the sample.

16. The method of claim 14 wherein the enzyme is a hydrolytic enzyme selected from a protease, a nuclease or a reductase.

17. The method of claim 14 wherein the enzyme is selected from the group consisting of the gene product of pep A1 (MCAP_{—}0157), pepA2 (MCAP_{—}0195), pepA (leucyl aminopeptidase, such as MHP7448_{—}0464), MCAP_{—}0267 (metalloendopeptidase), pepP (Xaa-Pro endopeptidase, such as MCAP_{—}0341 or MHP7448_{—}0649), MCAP_{—}0509, mapP (methionine amino peptidase, MCAP_{—}0675 or MHP7448_{—}0173), and mixtures thereof.

18. The method of claim 14 wherein the enzyme is selected from the group consisting of the 5′-3′ exonuclease encoded by MCAP_{—}0047 or MHP7448_{—}0581, the gene product of nfo (such as MCAP_{—}0060 or MHP7448_{—}0062), vacB (such as MCAP_{—}0097 or MHP7448_{—}0037), uvrC (such as MCAP_{—}0252 or MHP7448_{—}0066), mc (ribonuclease III, such as MCAP_{—}0492 or MHP7448_{—}0398), MCAP_{—}0768, uvrB (such as MCAP_{—}0773 or MHP7448_{—}0648), uvrA (such as MCAP_{—}0774 or MHP7448_{—}0091) and mixtures thereof.
